(19) United States
(12) Patent Application Publication
(10) Pub. No.: US 2015/0100037 A1
(43) Pub. Date: Apr. 9, 2015

(54) SYSTEM AND METHOD FOR PROVIDING ANIMAL MAINTENANCE

(71) Applicant: David R. Allsup, DALLAS, TX (US)

(72) Inventor: David R. Allsup, DALLAS, TX (US)

(21) Appl. No.: 14/048,653

(22) Filed: Oct. 8, 2013

(51) Int. Cl. A01K 13/00; A01K 1/035; A01K 1/06; A61B 19/00; G01B 5/00; G01B 17/00; G01N 25/56; A61M 5/178; A01K 11/00; G01B 11/00

(52) U.S. Cl. CPC A01K 13/001; A01K 15/026; A01K 1/0356; A01K 13/002; A01K 13/003; A01K 1/06; G01B 11/00; G01B 5/00; G01B 17/00; G01N 25/56; A61M 5/178; A61B 19/22; Y10S 901/41; Y10S 901/46; Y10S 901/09; A61M 2250/00
USPC 604/503; 119/608; 119/671; 119/751; 119/709; 119/51.01; 604/95.01; 901/41; 901/46; 901/9

(57) ABSTRACT

A system for providing animal maintenance includes a housing operable to accommodate an animal, an actuating arm coupled to the housing and to an attachment device, the attachment device is operable to perform one or more animal maintenance tasks, and the actuating arm is operable to apply the attachment device to the animal, a position sensor operable to determine the position of the animal relative to the position sensor, a restraint is operable to restrict the movement of the animal within the housing, and a processor communicatively coupled to the position sensor, the actuating arm, and the attachment device, the processor is operable to receive measurements from the position sensor and, in response to the received measurements, direct the actuating arm to apply the attachment device to the animal.

### TECHNICAL FIELD

This disclosure relates generally to a system for performing animal maintenance.

### BACKGROUND

Animal maintenance often includes performing various tasks to maintain an animal's health, appearance, disposition, and usefulness. For example, animal caregivers may regularly wash, groom, exercise, and provide medical care to animals in their care. Animal maintenance tasks may anger, stress, frighten, or otherwise agitate an animal making it difficult for a caregiver to safely perform animal maintenance tasks. Additionally, animal maintenance tasks are often time consuming, difficult, unsanitary, and/or unsafe.

### SUMMARY OF EXAMPLE EMBODIMENTS

According to embodiments of the present disclosure, disadvantages and problems associated with providing animal maintenance may be reduced or eliminated.

In certain embodiments, a system for providing animal maintenance includes a housing operable to accommodate an animal, an actuating arm coupled to the housing and to an attachment device, wherein the attachment device is operable to perform one or more animal maintenance tasks, and the actuating arm is operable to apply the attachment device to the animal, a position sensor coupled to at least one from the set comprising: the housing, the actuating arm, and the attachment device, wherein the position sensor is operable to determine the position of the animal relative to the position sensor; a restraint coupled to at least one from the set comprising: the housing, the actuating arm, and the attachment device, wherein the restraint is operable to restrict the movement of the animal within the housing; and a processor communicatively coupled to the position sensor, the actuating arm, and the attachment device, wherein the processor is operable to receive measurements from the position sensor and, in response to the received measurements, direct the actuating arm to apply the attachment device to the animal.

In an embodiment, a system for providing animal maintenance includes one or more actuating arms and one or more attachment devices operable to perform one or more animal maintenance tasks remotely, thereby protecting animal caregivers from injury and/or unsanitary conditions when performing animal maintenance tasks.

In particular embodiments, a system for providing animal maintenance includes one or more actuating arms and one or more attachment devices operable to perform one or more animal maintenance tasks autonomously, thereby protecting animal caregivers from injury and/or unsanitary conditions when performing animal maintenance tasks.

In an embodiment, a system for providing animal maintenance includes housing capable of configuring to accommodate animals of different sizes, thereby allowing the system to accommodate a plurality of different animals and/or to accommodate animals as they grow.

In a further embodiment, a system for providing animal maintenance includes a housing coupled to a restraint operable to restrict the movement of an animal within the housing, thereby preventing the animal from escaping the housing.

In a still further embodiment, a system for providing animal maintenance includes a distraction mechanism connected to a housing operable to distract, pacify, and/or sooth an animal within the housing, thereby preventing the animal from becoming angered, stressed, frightened, or otherwise agitated and obstructing maintenance tasks and injuring itself and/or a caregiver.

Other technical advantages of the present disclosure will be readily apparent to one skilled in the art from the following figures, descriptions, and claims. Moreover, while specific advantages have been enumerated above, various embodiments may include all, some, or none of the enumerated advantages.

### BRIEF DESCRIPTION OF THE DRAWINGS

For a more complete understanding of the present disclosure and for further features and advantages thereof, reference is now made to the following description taken in conjunction with the accompanying drawings, in which:

FIG. 1 illustrates an example system for providing animal maintenance, according to certain embodiments of the present disclosure;

FIG. 2 illustrates an example of an actuating arm and attachment device from the system illustrated in FIG. 1, according to certain embodiments of the present disclosure; and

FIG. 3 illustrates a flow chart of an example method for providing animal maintenance, according to certain embodiments of the present disclosure.

### DETAILED DESCRIPTION

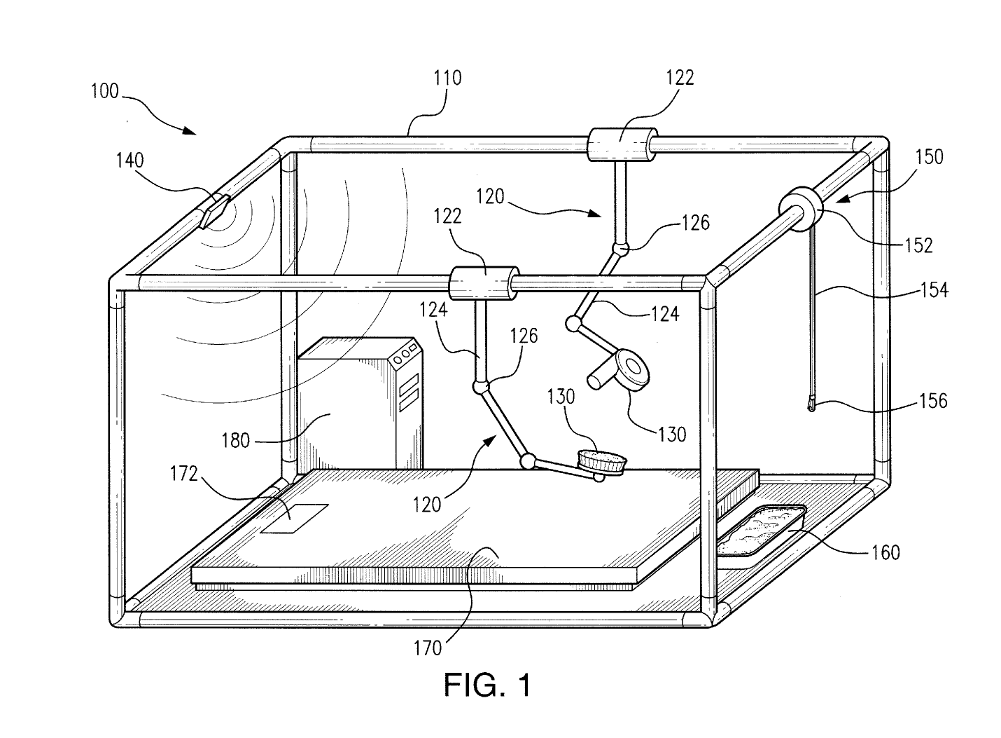

Embodiments of the present invention and its advantages are best understood by referring to FIGS. 1 through 3 of the drawings, like numerals being used for like and corresponding parts of the various drawings.

Animal caregivers must routinely perform animal maintenance tasks to maintain the health, appearance, disposition, and usefulness of animals in their care. For example, animal care givers may exercise, provide medical care, wash, groom, entertain, or otherwise maintain animals. Animal maintenance tasks can be time consuming, tedious, unsanitary, or otherwise unpleasant, and may anger, stress, frighten, or otherwise agitate the animals being maintained. It is often unsafe for both animals and caregivers to perform animal maintenance tasks when an animal is in an angered, stressed, frightened, or otherwise agitated state. Animal maintenance tasks include washing, applying conditioner, drying (e.g., air dryer and/or towel), cutting hair and/or fur, giving injections, applying medical instruments (e.g., thermometer), cooling (e.g., fan and/or mister), identifying parasites (e.g., by camera), removing parasites, shoeing, trimming claws, or any other task to maintain the health, appearance, disposition, and/or usefulness of an animal.

In certain embodiments, a system for providing animal maintenance includes a housing, one or more actuating arms, one or more attachment devices operable to couple to the actuating arms and perform animal maintenance tasks (e.g., a brush, comb, hose, nozzle, hair clippers, scissors, shears, air dryer, fan, towel, sponge, cloth, syringe, medicinal applicator, vacuum, claw trimmer, or any other tool suitable for performing animal maintenance tasks), one or more sensors operable to connect to one or more of the housing, the actuating arms, and the attachment devices, a restraint mechanism, a floor (e.g., a scale, a treadmill, or slotted floor), and a control module. The control module may receive from the one or more sensors position measurements and, based on the position measurements, maneuver one or more actuating arms to apply one or more attachment devices to an animal within the housing in order to perform a animal maintenance task.

FIG. 1 illustrates an example system for performing animal maintenance tasks. In an embodiment, system 100 includes housing 110, one or more actuating arms 120, one or more attachment devices 130, one or more sensors 140, one or more restraints 150, distraction mechanism 160, floor 170, and control module 180.

Housing 110 represents a structure operable to accommodate an animal that supports one or more components of system 100. Housing 110 may be comprised of any suitable material, for example, one or more of metal, wood, plastic, or fiberglass. In an embodiment, housing 110 is adjustable to accommodate animals of different sizes (e.g., cat, dog, sheep, cow, etc.). For example, housing 110 may be comprised of components operable to expand to different sizes (e.g., telescoping pipes), modular components that support adding or removing portions of housing 110 to increase or decrease the size (e.g., scaffolding), or other structure or material that allows housing 110 to dynamically increase or decrease in size.

In certain embodiments, the exterior of housing 110 may be configurable to comprise one or more different materials. For example, in one configuration, all or a portion of the exterior of housing 110 may be a lattice structure without walls (e.g., interconnected pipes or bars), in another configuration, all or a portion of the exterior of housing 110 may be comprised of a structurally solid material (e.g., wood, metal, plastic, and fiberglass), and in yet another configuration, all or a portion of the exterior of housing 110 may be comprised of a flexible material (e.g., cloth, flexible plastic, flexible fiberglass, or other flexible material). In an embodiment, the exterior of housing 110 includes a combination of two or more of the aforementioned exteriors (e.g., lattice, structurally solid, and/or visually solid). Different animals may benefit from different exterior configurations. For example, animals that become calm in dark confined areas may benefit from a structurally solid or flexible materials that limit the animal's field of view, animals that are a risk of injuring caretakers may benefit from a structurally solid materials, and animals that become agitated in confined spaces may benefit from the open lattice exterior, transparent solid, or transparent flexible materials.

In particular embodiments, housing 110 is coupled to one or more of actuating arm(s) 120, attachment device(s) 130, sensor(s) 140, restraint(s) 150, distraction mechanism(s) 160, floor 170, and control module 180. Housing 110 may also include attachment points operable to detachably connect to one or more of actuating arm(s) 120, attachment device(s) 130, sensor(s) 140, restraint(s) 150, distraction mechanism(s) 160, floor 170, and control module 180.

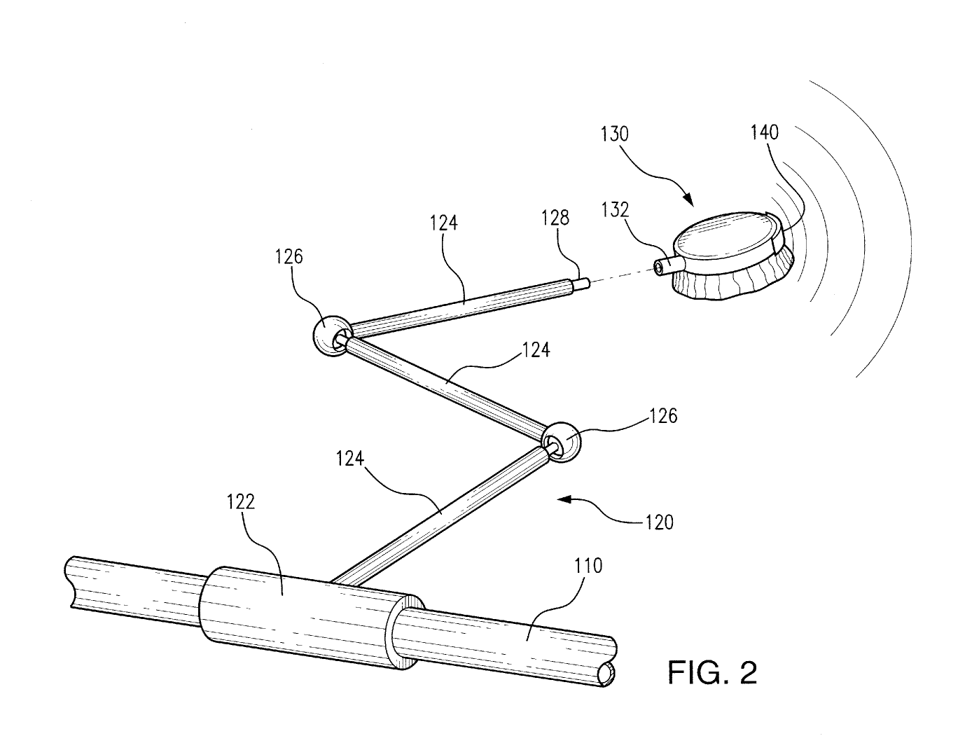

Actuating arm 120 represents a robotic arm operable to connect to housing 110 and to one or more attachment devices 130. In certain embodiments, actuating arm includes housing connection 122, rigid segments 124, and mechanical joints 126, and may communicatively couple to one or more different attachment devices 130. The components of actuating arm 120 are discussed in more detail below with respect to FIG. 2. Actuating arm 120 may be operable to communicatively couple to one or more attachment devices 130 in order to perform animal maintenance tasks. In certain embodiments, actuating arm 120 is operable to move on housing 110 (e.g., by cable, magnet, chain, wheel, or other component operable to move actuating arm 120). Actuating arm 120 may be coupled to one or more of a motor (e.g., electric or mechanical engine) and/or hoses (e.g., soap, water, conditioner, air, or other fluid used in animal maintenance tasks).

In certain embodiments, actuating arm 120 receives commands from control module 180. For example, control module 180 may detect that attachment device 130 is a brush (e.g., based on software on attachment device 130), and may determine the size and position of an animal within housing 110 (e.g., based on one more position sensors 140). In an embodiment, control module 180 receives position measurements from position sensors 140 and, based on the position measurements, communicates movement commands to actuating arm 120 to apply attachment device 130 (e.g., a brush) to the animal in order to perform the animal maintenance task of straightening the animal's fur.

Attachment device 130 represents a tool operable to couple to actuating arm 120 and perform animal maintenance tasks. Attachment device 130 may be a brush, comb, hose, nozzle, hair clippers, scissors, shears, air dryer, fan, towel, sponge, cloth, syringe, medicinal applicator, vacuum, claw trimmer, or any other tool suitable for performing animal maintenance tasks. In certain embodiments, attachment device 130 includes one or more memories, databases, interfaces, and/or processors. Attachment device 130 may include logic (e.g., software) operable to instruct control module 180 on how to operate attachment device 130. Attachment device 130 may have a connector to connect to actuating arm 120. In an embodiment, attachment device 130 includes a communication port (e.g., universal serial bus (USB) port) operable to communicatively couple to actuating arm 120. Attachment device 130 may further include one or more physical, electrical, hydraulic, pneumatic, data, or other suitable connections between actuating arm 120 and attachment device 130. In certain embodiments, attachment device includes one or more sensors 140. Attachment device 120 is discussed in more detail below with respect to FIG. 2.

Sensors 140 represent one or more devices operable to measure physical properties. Sensors 140 may measure position, distance, motion, connectivity, moisture, temperature, pressure, various wavelengths of light (e.g., infrared, visible, and/or ultraviolet), various wavelengths of sound (e.g., ultrasonic and/or infrasonic) particle concentration (e.g., contaminants in water), or any other measurement suitable for animal maintenance tasks. In certain embodiments, sensor 140 is a camera and is operable to detect visible, infrared, and/or ultraviolet light. Sensors 140 may be operable to identify parasites (e.g., fleas, ticks, worms, or other parasite), for example, using a camera with a magnifying lens. In an embodiment, sensor 140 is an ultrasonic sensor that operates in ranges greater than 80 kHz to avoid the hearing range of most mammals (e.g., cats, dogs, and people).

Sensors 140 may couple to housing 110, actuating arm 120, attachment device 130, restraint 150, distraction mechanism 160, and/or floor 170. In certain embodiments, sensor 140 includes one or more memories, databases, interfaces, and/or processors. Sensors 140 may include logic (e.g., software) operable to instruct control module 180 on how to operate sensors 140. Sensors 140 may include a communication port (e.g., USB port) operable to communicatively couple to one or more of housing 110, actuating arm 120, attachment device 130, restraint 150, distraction mechanism 160, and/or floor 170 (e.g., by a communication port such as USB).

Restraint 150 represents an apparatus operable to restrict the movement of an animal within housing 110. Restraint 150 may include one or more of a leash, lead, harness, blinder, immobilize, cuff, chain, ring, barrier, yoke, and/or any other suitable apparatus operable to restrict the movement of an animal within housing 110. In certain embodiments, restraint 150 includes retractor 152, tether 154, restraint connector 156, and/or sensor 140. Refractor 152 represents a component of restraint 150 operable to retract or extend tether 154. In certain embodiments, retractor 152 includes a spring mechanism that keeps tether 154 taut or retracted. Retractor 152 may include a locking mechanism operable to lock tether 154 so that it cannot extend. In certain embodiments, the tension applied by retractor 152 on tether 154 is controlled by control module 180. For example, control module 180 may control retractor 152 to move an animal within housing 110 (e.g., lift an animal's head up). Tether 154 represents a connection between housing 110 and restraint connector 156 (e.g., leash or lead). Tether 154 may be comprised of any suitable material including cloth, leather, metal, plastic, fiberglass, and/or cable. Restraint connector 156 represents a component of restraint 150 operable to connect tether 154 to an animal (e.g., to a harness, collar, yoke, and/or ring). In certain embodiments, restraint connector 156 includes a clip, clasp, clamp, or other suitable apparatus operable to securely connect tether 154 to an animal.

Distraction mechanism 160 represents a component of system 100 operable to distract, calm, pacify, and/or sooth an animal within housing 110. Distraction mechanism 160 may include one or more of food (e.g., a food dispenser), toys (e.g., chew toys), a light emitter (e.g., a laser dot), an audio player (e.g., a white noise emitter, simulated heartbeat), a scent emitter (e.g., scent masker), and/or vision blinder (e.g., blinder, blindfold, or hood). In certain embodiments, distraction mechanism 160 is communicatively coupled to control module 180. Control module 180 may be operable to control the operation of distraction mechanism 160 (e.g., controlling sound from sound emitter, controlling light from light emitter, controlling airflow around scent emitter, manipulating toys, and refilling food containers).

Floor 170 represents a base for an animal within housing 110. Floor 170 may include one or more of a scale, treadmill, slotted floor (e.g., to allow animal waste, cleaning fluids, water, fur, or other waste material to drain out of housing 110), or any other suitable base. In certain embodiments, floor 170 includes display 172 operable to display information about floor 170 (e.g., display from a scale or treadmill). Floor 170 may be communicatively coupled to control module 180. In an embodiment, control module 180 is operable to control the operation of floor 170 (e.g., speed of a treadmill, incline of a treadmill, and units of a scale). Floor 170 may be comprised of a material that prevents animals from slipping (e.g., rubber, plastic, fiberglass, or other suitable non-slip material).

Control module 180 represents a device operable to control the operation of one or more components of system 100 (e.g., actuating arm(s) 120, attachment device(s) 130, sensor(s) 140, restraint 150, distraction mechanism 160, and/or floor 170). Control module 180 may be operable to support remote and/or autonomous executing of animal maintenance tasks using actuating arm(s) 120 and attachment device(s) 130. For example, control module 180 may be operable to receive remote signals from an operator (e.g., animal caregiver) and, based on the received signals, maneuver one or more actuating arms 120 and one or more attachment devices 130 to perform an animal maintenance task (e.g., clean an animal). In certain embodiments, control module 180 includes software that enables control module 180 to autonomously control one or more actuating arms 120 and one or more attachment devices 130 to perform an animal maintenance task (e.g., clean an animal). Control module 180 may include one or more processors, memories, interfaces, and/or databases. In certain embodiments, control module 180 is a computer, tablet, smart phone, microprocessor, and/or application specific integrated circuit (ASIC).

Components of system 100 (e.g., actuating arm(s) 120, attachment device(s) 130, sensor(s) 140, restraint 150, distraction mechanism 160, floor 170, and control module 180) may communicate over one or more networks. In certain embodiments, a network includes any interconnecting system capable of transmitting audio, video, electrical signals, optical signals, data, messages, or any combination of the preceding. A network may include all or a portion of a public switched telephone network (PSTN), a public or private data network, a local area network (LAN), a metropolitan area network (MAN), a wide area network (WAN), a local, regional, or global communication or computer network, such as the Internet, a wireline or wireless network, an enterprise intranet, or any other suitable communication link, including combinations thereof, operable to facilitate communication between the components of system 100. A network may utilize any suitable communication protocol operable to support communication between one or more of actuating arm(s) 120, attachment device(s) 130, sensor(s) 140, restraint 150, distraction mechanism 160, floor 170, and control module 180, including Ethernet over a local area network (LAN), Bluetooth, and IEEE 802.11.

A module (e.g., control module 180) may execute any suitable operating system such as IBM's zSeries/Operating System (z/OS), MS-DOS, PC-DOS, MAC-OS, WINDOWS, a .NET environment, UNIX, OpenVMS, or any other appropriate operating system, including future operating systems. The functions of a module may be performed by any suitable combination of one or more servers or other components at one or more locations. In embodiments where modules represent a server, the server may be a private server, and the server may be a virtual or physical server. Additionally, a module may include any suitable component that functions as a server.

Components of system 100, such as actuating arm(s) 120, attachment device(s) 130, sensor(s) 140, restraint 150, distraction mechanism 160, floor 170, and control module 180 may include one or more processors, interfaces, memories, and/or databases. A processor represents any computing device configured to control the operation of one or more components of system 100. A processor may comprise one or more processors and may be a programmable logic device, a microcontroller, a microprocessor, any suitable processing device, or any suitable combination of the preceding. A processor includes any hardware or software that operates to control and process information received by a component of system 100. In certain embodiments, a processor communicatively couples to other components of system 100, such as a module (e.g., control module 180), an interface, a memory, a database, or any other suitable component.

An interface represents any device operable to receive input, send output, process the input or output, or perform other suitable operations for a component of system 100. An interface includes any port or connection, real or virtual, including any suitable hardware or software, including protocol conversion and data processing capabilities, to communicate through a network. In certain embodiments, an interface includes a user interface (e.g., physical input, graphical user interface, touchscreen, buttons, switches, transducer, or any other suitable method to receive input from a user).

A memory represents any device operable to store, either permanently or temporarily, data, operational software, or other information for a processor. Memory includes any one or a combination of volatile or non-volatile local or remote devices suitable for storing information. For example, a memory may include random access memory (RAM), read only memory (ROM), magnetic storage devices, optical storage devices, semiconductor storage devices, or any other suitable information storage device or a combination of these devices. A memory may include any suitable information for use in the operation of component of system 100. A memory may further include some or all of one or more databases.

Logic may perform the operation of any component of system 100, for example, logic executes instructions to generate output from input. Logic may include hardware, software, or other logic. Logic may be encoded in one or more non-transitory, tangible media, such as a computer-readable medium or any other suitable tangible medium, and may perform operations when executed by a computer or processor. Certain logic, such as a processor, may manage the operation of a component.

In an embodiment of operation, control module 180 determines whether an animal is within housing 110, for example, based on measurements from position sensors 140 and/or sensors 140 on restraint 150. Control module 180 may be able to determine the type of animal within housing 110, for example, based on sensors 140 (e.g., cameras) and/or user input through an interface of control module 180 (e.g., a graphical user interface). In an embodiment, control module 180 determines an animal maintenance task to perform, for example, based on one or more of user input through an interface of control module 180, sensor 140 measurements, and/or information communicated by one or more attachment devices 130 (e.g., identification information of attachment device 130 and/or control instructions for attachment device 130). Control module 180 may maneuver one or more actuating arms 120 coupled to attachment devices 130 based on one or more received sensor 140 measurements to apply attachment devices 130 to an animal within housing 110 in order to perform an animal maintenance task (e.g., applying a hose to an animal to clean it, measuring rinse water to determine when the animal is clean, applying an air dryer to the animal to dry it, measuring the moisture on the animal to determine when it is dry, and applying a brush to the animal to straighten its fur).

Modifications, additions, or omissions may be made to system 100. System 100 may include more, fewer, or other components. Any suitable component of system 100 may include a processor, interface, logic, memory, or other suitable element. System 100 may be configured to accommodate any suitable animal maintenance tool (e.g., as attachment device 130).

FIG. 2 illustrates an example of an actuating arm 120 and attachment device 130 from the system illustrated in FIG. 1. FIG. 2 includes housing 110, actuating arm 120, attachment device 130, and sensor 140. As discussed above with respect to FIG. 1, actuating arm 120 represents a robotic arm operable to connect to housing 110 and to one or more attachment devices 130. In the illustrated embodiment, actuating arm 120 includes housing connection 122, rigid segments 124, mechanical joints 126, and attachment device connection 128.

Housing connection 122 represents a connector that couples actuating arm 120 to housing 110. Housing connection 122 may be comprised of any suitable material (e.g., metal, plastic, wood, fiberglass, rubber, or any combination of the foregoing) and may be any suitable type of connector (e.g., clamp, slide, band, latch, etc.). In certain embodiments, housing connection 122 is operable to move along housing 110 allowing actuating arm 120 to reach different portions of an animal within housing 110. For example, housing connection 122 may be connected to a track, cable, magnets, chain, wheels, or other component operable to move housing connection 122 along housing 110. In certain embodiments, housing connection 122 includes a mechanical joint 126.

Rigid segments 124 represent a rigid structure operable to connect one or more mechanical joints 126 of actuating arm 120 to one or more of housing connection 122, mechanical joints 126, and attachment device connection 128. Rigid segments 124 may be of any suitable material, for example, plastic, fiberglass, metal, or any combination of the foregoing. In certain embodiments, rigid segments 124 include mechanical, electrical, hydraulic, and/or pneumatic components operable to enable actuating arm 120 to maneuver attachment device 130. Rigid segments 124 may be extendable and retractable (e.g., telescopic).

Mechanical joints 126 represent joints between one or more of housing connection 122, rigid segments 124, and attachment device connection 128. Mechanical joints 126 may be any suitable type of mechanical joint including spherical, planer, cylindrical, revolute, prismatic, and screw. In certain embodiments, mechanical joints 126 have multiple degrees of freedom.

Attachment device connection 128 represents connection between attachment device 130 and actuating arm 120. Attachment device connection 128 may be physical, electrical, hydraulic, pneumatic, data, or other suitable connection between actuating arm 120 and attachment device 130.

In certain embodiments, housing 110, actuating arm 120, and/or attachment device 130 include one or more sensors 140. As discussed above with respect to FIG. 1, sensor 140 may measure position, distance, motion, connectivity, moisture, temperature, pressure, various wavelengths of light (e g, infrared, visible, and/or ultraviolet), various wavelengths of sound (e.g., ultrasonic and/or infrasonic) particle concentration (e.g., contaminants in water), or any other measurement suitable for animal maintenance tasks. In certain embodiments, sensor(s) 140 couple to one or more of housing 110, actuating arm 120, attachment device 130, restraint 150, distraction mechanism 160, and floor 170.

In particular embodiments, attachment device(s) 130 include logic (e.g., software) operable to communicate with control module 180. For example, when attachment device 130 connects to actuating arm 120, logic within actuating device 130 may communicate to control module 180 information including: that attachment device 130 is connected, identification information related to attachment device 130, information related to the operation of attachment device 130, or any other suitable information. In certain embodiments, the logic in attachment device 130 includes a software driver.

Modifications, additions, or omissions may be made to actuating arm 120 and/or attachment device 130. Actuating arm 120 and/or attachment device 130 may include more, fewer, or other components. Any suitable component of actuating arm 120 and/or attachment device 130 may include a processor, interface, logic, memory, sensor 140, or other suitable elements.

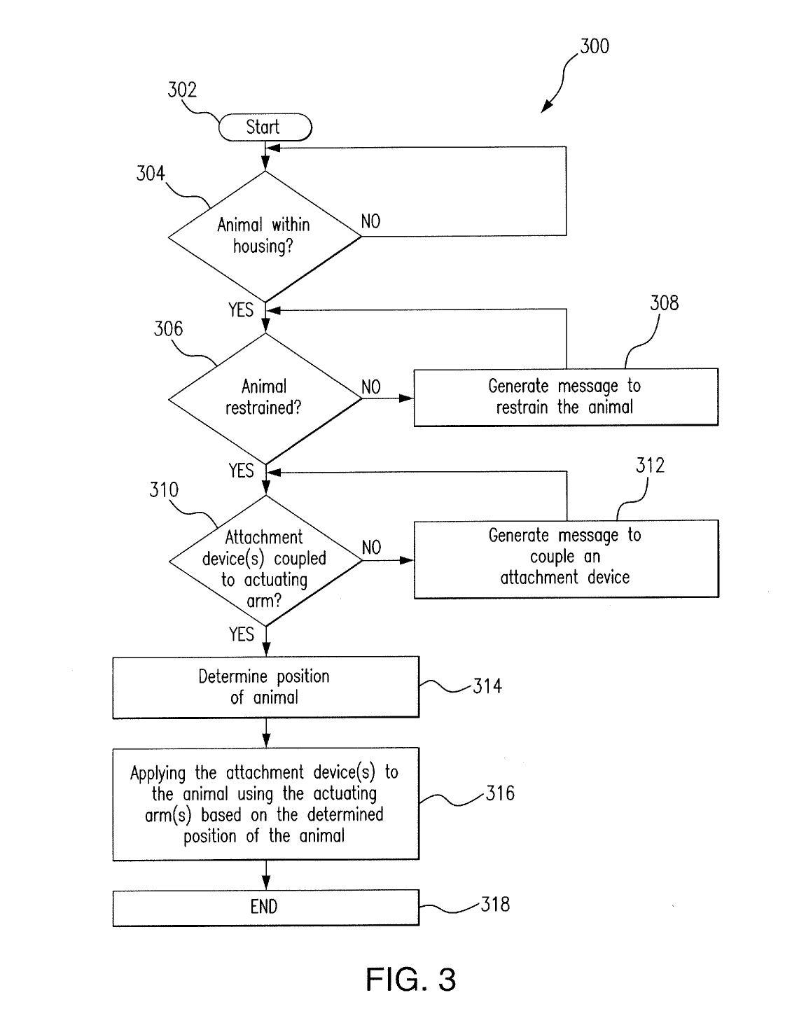

FIG. 3 illustrates a flow chart of an example method for providing animal maintenance tasks. Method 300 begins at step 302. At step 304, it is determined whether an animal is within housing 110. For example, one or more sensors 140 (e.g., position sensor 140) may detect that an animal is within housing 110. If no animal is detected within housing 110, the method returns to step 304 to continue to determine whether an animal is within housing 110. If an animal is detected within the housing, the method moves to step 306. At step 306, it is determined whether the animal is connected to restraint 150 within housing 110. For example, restraint 150 and/or sensors 140 may detect that restraint 150 is connected to an animal. In certain embodiments, restraint 150 and/or sensors 140 are operable to communicate whether an animal is connected to restraint 150 to control module 180. If it is determined that the animal is connected to restraint 150, at step 308 a message is generated (e.g., by control module 180) requesting that the animal be connected to restraint 150 and the method returns to step 306 to continue to determine whether the animal is connected to restraint 150. If it is determined that the animal is connected to restraint 150, the method moves to step 310.

At step 310 it is determined whether one or more attachment devices 130 are connected to one or more actuating arms 120. For example, attachment devices 130 and/or actuating arms 120 may be operable to detect when attachment devices 130 are connected to actuating arms 120. In an embodiment, attachment devices 130 and/or actuating arms 120 are operable to communicate whether attachment device 130 is connected to actuating arm 120 to control module 180. If it is determined that attachment device 130 is not connected to actuating arm 120, the method moves to step 312 and a message is generated (e.g., by control module 180) requesting that attachment device 130 be connected to actuating arm 120 and the method returns to step 310. If it is determined that attachment device 130 is connected to actuating arm 120, the method moves to step 314.

At step 314 the position of the animal within housing 110 is determined. For example, sensors 140 (e.g., position sensors 140) may determine the position of the animal within the housing and communicate that information to control module 180. At step 316, attachment device 130 is applied to the animal within housing 110 with actuating arm 120 to perform an animal maintenance task. For example, if attachment device 130 is a brush, control module 180 may apply the brush to the animal with actuating arm 120 in order to perform the animal maintenance task of straightening the animal's fur. In certain embodiments, control module 180 utilizes updating position measurements from sensor(s) 140 to direct actuating arm(s) 120 in applying attachment device(s) to the animal. At step 318 the method ends.

Modifications, additions, or omissions may be made to method 300. The method may include more, fewer, or other steps. Additionally, steps may be performed in any suitable order, in parallel, and/or sequentially. Any suitable component of may perform one or more steps of method 300.

Certain embodiments of the present disclosure may provide one or more technical advantages having specific technical effects. Other technical advantages of the present disclosure will be readily apparent to one skilled in the art from the following figures, descriptions, and claims. Moreover, while specific advantages have been enumerated, various embodiments may include all, some, or none of the enumerated advantages.

In an embodiment, a system for providing animal maintenance includes one or more actuating arms and one or more attachment devices operable to perform one or more animal maintenance tasks remotely, thereby protecting animal caregivers from injury and/or unsanitary conditions when performing animal maintenance tasks.

In particular embodiments, a system for providing animal maintenance includes one or more actuating arms and one or more attachment devices operable to perform one or more animal maintenance tasks autonomously, thereby protecting animal caregivers from injury and/or unsanitary conditions when performing animal maintenance tasks.

In an embodiment, a system for providing animal maintenance includes housing capable of configuring to accommodate animals of different sizes, thereby allowing the system to accommodate a plurality of different animals and/or to accommodate animals as they grow.

In a further embodiment, a system for providing animal maintenance includes a housing coupled to a restraint operable to restrict the movement of an animal within the housing, thereby preventing the animal from escaping the housing.

In a still further embodiment, a system for providing animal maintenance includes a distraction mechanism connected to a housing operable to distract, pacify, and/or sooth an animal within the housing, thereby preventing the animal from becoming angered, stressed, frightened, or otherwise agitated and obstructing maintenance tasks and injuring itself and/or a caregiver.

Other technical advantages of the present disclosure will be readily apparent to one skilled in the art from the following figures, descriptions, and claims. Moreover, while specific advantages have been enumerated above, various embodiments may include all, some, or none of the enumerated advantages.

Although the present disclosure has been described in several embodiments, a myriad of changes, variations, alterations, transformations, and modifications may be suggested to one skilled in the art, and it is intended that the present disclosure encompass such changes, variations, alterations, transformations, and modifications as fall within the scope of the appended claims.

What is claimed is:

1. A system for providing animal maintenance, comprising:
an adjustable housing operable to configure to different dimensions to accommodate animals of different sizes;
an actuating arm coupled to the housing and detachably coupled to an attachment device, wherein the attachment device is operable to perform one or more animal maintenance tasks, the actuating arm is operable to apply the attachment device to an animal within the housing, and the attachment device is one from the set comprising: a brush, a hose, a clipper, a scissor, a hot air dryer, a fan, a towel, a syringe, a medicinal applicator, or a vacuum;
a position sensor communicatively coupled to at least one from the set comprising: the housing, the actuating arm, and the attachment device, wherein the position sensor is operable to determine the position of the animal relative to the position sensor, and the position sensor is one from the set comprising: an ultrasonic sensor, an infrared sensor, a camera, a pressure sensor, and a laser;
a moisture sensor communicatively coupled to at least one from the set comprising: the housing, the actuating arm, and the maintenance device, wherein the moisture sensor is operable to measure an amount of moisture on the animal;
a distraction mechanism coupled to at least one from the set comprising: the housing, the actuating arm, and the maintenance device, wherein the distraction mechanism is operable to distract the animal while it is within the housing, and the distraction mechanism is at least one from the set comprising: a food dispenser, a chew toy, a light emitter, an audio player, a scent emitter, and a vision blinder;
a restraint coupled to at least one from the set comprising: the housing, the actuating arm, and the maintenance device, wherein the restraint is operable to restrict the movement of the animal within the housing;
a floor, wherein the floor includes at least one from the set comprising: a scale, a treadmill, and a slotted floor; and
a processor communicatively coupled to the actuating arm, the attachment device, the position sensor, and the moisture sensor, wherein the processor is operable to receive measurements from the position sensor and the moisture sensor and, in response to the received measurements, direct the actuating arm to apply the actuating device to the animal to perform one or more animal maintenance tasks.

2. The system of claim 1, wherein the position sensor is an ultrasonic sensor and the frequency broadcasted by the ultrasonic sensor is not less than 80 kHz.

3. A system for providing animal maintenance, comprising:
a housing operable to accommodate an animal;
an actuating arm coupled to the housing and to an attachment device, wherein the attachment device is operable to perform one or more animal maintenance tasks, and the actuating arm is operable to apply the attachment device to the animal;
a position sensor coupled to at least one from the set comprising: the housing, the actuating arm, and the attachment device, wherein the position sensor is operable to determine a position of the animal relative to the position sensor;
a restraint coupled to at least one from the set comprising: the housing, the actuating arm, and the attachment device, wherein the restraint is operable to restrict the movement of the animal within the housing; and
a processor communicatively coupled to the position sensor, the actuating arm, and the attachment device, wherein the processor is operable to receive measurements from the position sensor and, in response to the received measurements, direct the actuating arm to apply the attachment device to the animal.

4. The system of claim 3, wherein the position sensor is one from the set comprising: an ultrasonic sensor, an infrared sensor, a camera, a pressure sensor, and a laser.

5. The system of claim 4, wherein the position sensor is an ultrasonic sensor and the frequency broadcasted by the ultrasonic sensor is not less than 80 kHz.

6. The system of claim 3, further comprising:
a moisture sensor communicatively coupled to the processor and coupled to at least one from the set comprising: the housing, the actuating arm, and the attachment device, wherein the moisture sensor is operable to measure an amount of moisture on the animal.

7. The system of claim 3, further comprising a distraction mechanism coupled to at least one from the set comprising: the housing, the actuating arm, and the attachment device, wherein the distraction mechanism is operable to distract the animal, and the distraction mechanism is at least one from the set comprising: a food dispenser, a chew toy, a light emitter, an audio player, a scent emitter, and a vision blinder.

8. The system of claim 3, further comprising a floor coupled to the housing, wherein the floor includes at least one from a set comprising: a scale, a treadmill, and a slotted floor.

9. The system of claim 3, wherein the housing is adjustable to configure to accommodate animals of different sizes.

10. The system of claim 3, wherein the attachment device is one from the set comprising: a brush, a hose, a clipper, a scissor, a hot air dryer, a fan, a towel, a syringe, a medicinal applicator, or a vacuum.

11. The system of claim 3, wherein the attachment device is detachably coupled to the actuating arm and the actuating arm is operable to detachably couple to a plurality of different attachment devices.

12. A method for providing animal maintenance, comprising:
determining that an animal is within a housing;
determining that the animal is connected to a restraint coupled to at least one from a set comprising: an actuating arm and an attachment device;
determining, by a position sensor, a position of the animal relative to the position sensor, wherein the position sensor is communicatively coupled to at least one from a set comprising: the housing, the actuating arm, and the attachment device;
receiving, by a processor communicatively coupled to the position sensor, measurements relating to the position of the animal with respect to the position sensor; and
applying, by the processor, the attachment device to the animal using the actuating arm to perform an animal maintenance task.

13. The method of claim 12, wherein the position sensor is one from a set comprising: an ultrasonic sensor, an infrared sensor, a camera, a laser, and a pressure sensor.

14. The method of claim 13, wherein the position sensor is an ultrasonic sensor and the frequency broadcasted by the ultrasonic sensor is not less than 80 kHz.

15. The method of claim 12, further comprising measuring, by a moisture sensor, an amount of moisture on the animal, wherein the moisture sensor is communicatively coupled to the processor and coupled to at least one from a set comprising: the housing, the actuating arm, and the grooming mechanism.

16. The method of claim 12, further comprising distracting the animal using a distraction mechanism, wherein the distraction mechanism is at least one from a set comprising: a food dispenser, a chew toy, a light emitter, an audio player, a scent emitter, and a vision blinder.

17. The method of claim 12, further comprising a floor coupled to the housing, wherein the floor is one from a set comprising: a scale, a treadmill, or a slotted floor.

18. The method of claim 12, further comprising adjusting the housing to configure to accommodate animals of different sizes.

19. The method of claim 12, wherein the attachment device is one from a set comprising: a brush, a hose, a clipper, a scissor, a hot air dryer, a fan, a towel, a syringe, a medicinal applicator, or a vacuum.

20. The method of claim 12, wherein the attachment device is detachably coupled to the actuating arm and the actuating arm is operable to detachably couple to a plurality of different attachment devices.
